(19) United States
(12) Plant Patent Application Publication
(10) Pub. No.: US 2015/0289430 P1
(43) Pub. Date: Oct. 8, 2015

(54) Strawberry plant named 'NJ08-08-6'

(50) ;

(71) Applicant: Rutgers, The State University, New Brunswick, NJ (US)

(72) Inventors: Gojko J. Jelenkovic, Piscataway, NJ (US); Peter J. Nitzsche, Long Valley, NJ (US); William T. Hlubik, Bordentown, NJ (US); Jessica D. Baculis, Watchung, NJ (US)

(73) Assignee: Rutgers, The State University, New Brunswick, NJ (US)

(21) Appl. No.: 13/999,929

(22) Filed: Apr. 4, 2014

(51) Int. Cl. A01H 5/00

(52) U.S. Cl. PLT/208

(57) ABSTRACT

A new and distinct cultivar of strawberry plant (Fragaria×annanassa), was developed from seed produced by a hand pollinated cross between ‘NJ01-73-3’ and ‘NJ03-232-2’. The new strawberry plant named ‘NJ08-08-6’ is distinguished by its highly vigorous, upright growth habit, high productivity, excellent fruit quality and exquisite flavor.

### CROSS REFERENCE TO RELATED APPLICATIONS

NONE

### STATEMENT REGARDING FEDERALLY SPONSORED RESEARCH OR DEVELOPMENT

NONE

### LATIN NAME OF GENUS AND SPECIES OF THE PLANT CLAIMED

Fragaria annanassa

### VARIETY DENOMINATION

‘NJ08-08-6’

### BACKGROUND OF THE NEW PLANT

A new and distinct cultivar of strawberry named ‘NJ08-08-6’ is a short day cultivar similar to ‘Chandler’ (U.S. Plant Pat. No. 5,262) but it is distinguished by its elongated petioles, upright plant growth and large fruits, with a uniform red color, shiny in appearance and conical in shape with exceptional flavor. The cultivar is well adapted to high density plasti-culture growing systems and has been shown to perform well in the eastern United States. This new variety should be of commercial value, particularly for farmers with direct markets, due to its unique growth characteristics and excellent fruit production and flavor.

### ORIGIN OF THE VARIETY

This new strawberry genotype was developed from a controlled cross of ‘NJ01-73-3’ (unpatented) as the female parent and ‘NJ03-232-2’(unpatented) as the male parent. The female parent ‘NJ01-73-3’ was chosen for its vigorous growth habit, high productivity, large fruit size and excellent fruit firmness; however, its berries lacked color and flavor. The male parent ‘NJ03-232-2’ was chosen for the excellent flavor of its berries; however, it was a plant of moderate vigor and low productivity with small berries. Both parental genotypes are derivatives of many cycles of crosses and selection in the Rutgers breeding program. Seeds of the cross were germinated in a greenhouse and planted in the field at the NJAES research farm #3 in New Brunswick, N.J. in 2008. After the first round of selection, the plant was evaluated for two seasons in small observational plots. The selected plant designated as ‘NJ08-08-6’, was recognized as the best combination of parental phenotypes, including high vigor, high productivity, uniform fruit coloring and large flavorful fruit. The ‘NJ08-08-6’ plant was then asexually propagated for further evaluations 2012 to 2013 in observational and replicated trials in several locations in New Jersey, one location in North Carolina and one location in Maryland.

### SUMMARY OF THE VARIETY

The ‘NJ08-08-6’ plant is primarily adapted to the climate and conditions of the eastern United States (zones 5b, 6a & b, 7a & b, 8a & b and 9a) where it demonstrates vigorous, upright plant growth, resulting in observational reductions in strawberry pathogens. It is characterized by its high production of large, uniformly ripened, conical fruit, with exceptionally sweet and aromatic flavors.

### BRIEF DESCRIPTION OF THE PHOTOGRAPH

FIG. 1 illustrates the characteristics (shape, size and coloring) of typical ripened fruit from an ‘NJ08-08-6’ plant; and

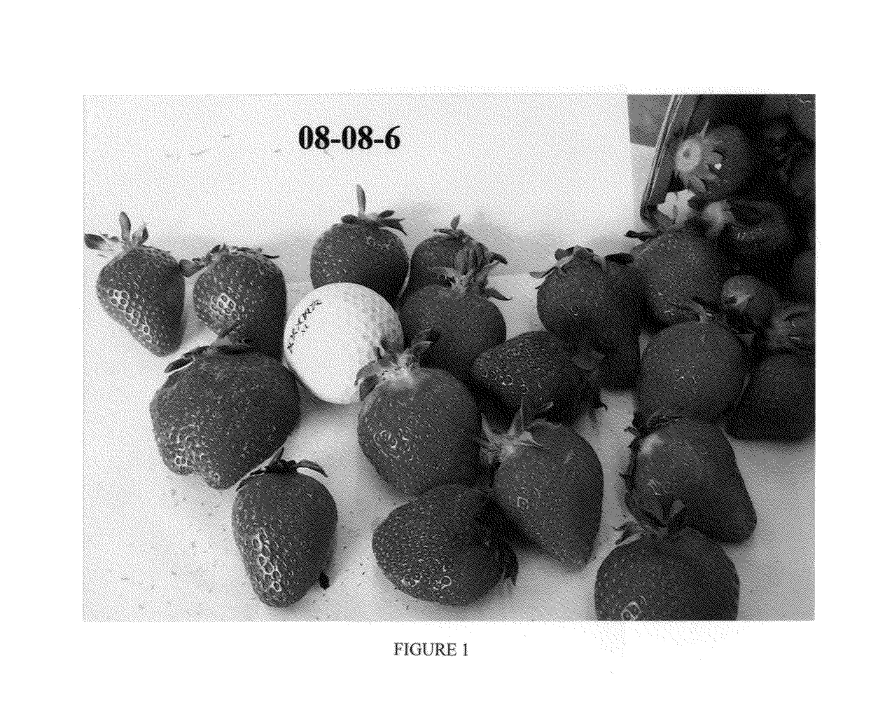

FIG. 2 illustrates the characteristic upright growth pattern of the mature petioles and foliage.

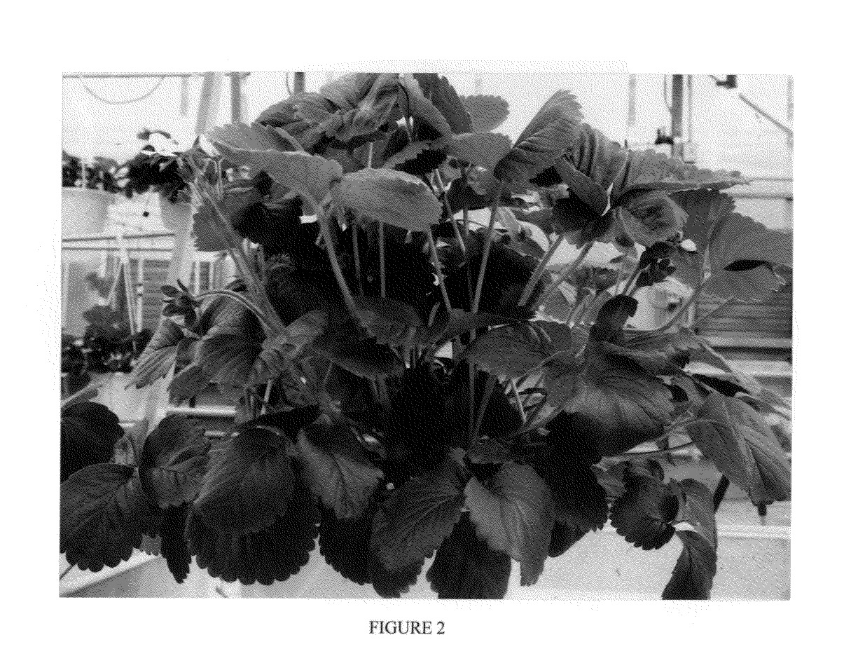

All color references below are measured against The Royal Horticultural Society Colour Chart (R.H.S. 1996 Ed.). Colors of foliage, fruit, inflorescence and other plant parts may vary from year to year and from location to location depending on horticultural practices, light conditions, air temperature, soil fertility, etc. CL DETAILED BOTANICAL DESCRIPTION

The ‘NJ08-08-6’ plant can be distinguished from other strawberry varieties by the structure of its foliar growth. In the spring as the leaves arise from the crown of the plant, there is a rapid elongation of the petioles causing an elevation of the leaf lobes and an upright mode of growth. The petioles are very sturdy and remain in an upright position for most of the growing season. The peduncles, which bear the fruit on the tips of their branches, have a more prostrate growth habit; because of the weight of the fruit, they often lay on the plastic around the crown. This particular morphological structuring on the ‘NJ08-08-6’ plant makes the lower parts of the bush more open to air movement making the environment less favorable for microorganisms that can cause fruit and leaf diseases.

During the two year observational field evaluations at Horticultural Farm 3 in New Brunswick, N.J., no pesticides or other protective measures were used. During this time, the plant displayed no visually detrimental symptoms of disease on its foliage or fruit.

The most distinctive feature of the ‘NJ08-08-6’ plant is the ripened fruits. The fruits are an appealing shiny red fruit, with a uniform conical shape. The fruits are only slightly longer than they are wide with a 1.11 length to width ratio. Their red color range is between RHS 44C and RHS 45C. They are smooth, solid and firm when handled and display some degree of resistance to fruit rot. The exceptional flavor of the fruit is the most distinguishing characteristic. The sweetness and aroma of the fruit supersedes the quality found in known commercially grown strawberry varieties. During the 2013 season strawberries from the ‘NJ08-08-6’ plants were compared with ‘Chandler’ by 20 panelists at one of the research locations. In this survey ‘NJJ08-08-6’ rated higher for sweetness, acidity and overall flavor compared to Chandler.

The time from flowering to fruit ripening (harvest) ranges from 28-34 days; which categorizes this genotype as a mid-season variety. Fruits turn red acropetally, from the fruit calyx toward the tip. Under particularly warm conditions in the spring, a primary fruit can become completely red in a single day; however, formation of anthogenesis, sugars, aromatics and other quality ingredients require at least 2 additional days.

TABLE 1
Flower Characteristics of NJAES strawberry
selections and cultivars, North Brunswick, NJ 2013

| Characteristic | ‘NJ08-08-6’ | Chandler | ‘NJ03-232-2’ |
|---|---|---|---|
| Flower color^{x} | White | White | White |
| Flower Head diameter | 2.90 | 4.8 | 3.8 |
| (mm)^{x} | | | |
| Petal Number^{x} | 4.85 | 5.6 | 5.1 |
| Corolla diameter (mm)^{x} | 23.90 | 26.15 | 26.50 |
| Calyx diameter (mm)^{x} | 17.25 | 21.15 | 23.65 |
| Sepal Number^{x} | 9.65 | 11.2 | 10.1 |

Data taken May 22 to Jun. 15, 2013
^{x}Means based on the average of 20 observations.

TABLE 2
Fruit Characteristics of NJAES strawberry selections
and cultivars, North Brunswick,NJ 2013

| | | | | Earliglow |
|---|---|---|---|---|
| | | | | (Unpat- |
| | ‘NJ08-08-6’ | Chandler | ‘NJ03-232-2’ | ented) |
| Fruit Length (mm)^{x} | 33.6545 | 31.04 | 36.14 | 23.69 |
| Fruit Width (mm)^{x} | 27.12 | 26.41 | 32.76 | 22.77 |
| Length/Width Ratio^{x} | 1.24 | 1.18 | 1.11 | 1.04 |
| % Soluble Solids | 8.28 | 8.26 | 8.84 | 10.49 |
| (Brix)^{y} | | | | |
| Fruit Density (g)^{x} | 167.9 | 172.6 | 137.23 | 128 |
| Average Berry | 11.29 | 12.37 | 11.8 | 8.59 |
| Weight (g)^{x} | | | | |

Data taken May 17 to Jun. 15, 2013
^{x}Means based on the average of 20 observations.
^{y}Means based on the average of 10 observations.

We claim:

1. A new and distinct variety of strawberry plant, designated ‘NJ08-08-6’ substantially as herein shown and described.
